(19) United States
(12) Patent Application Publication
(10) Pub. No.: US 2017/0224214 A1
(43) Pub. Date: Aug. 10, 2017

(54) INTEROPERABLE WEARABLE DEVICES AND COMMUNICATION PLATFORM

(71) Applicants: Michael Saigh, Clayton, MO (US); Xiaohong Zhang, Clayton, MO (US); Andrew Saigh, Clayton, MO (US); Susan Carpenter, Clayton, MO (US)

(72) Inventors: Michael Saigh, Clayton, MO (US); Xiaohong Zhang, Clayton, MO (US); Andrew Saigh, Clayton, MO (US); Susan Carpenter, Clayton, MO (US)

(21) Appl. No.: 14/754,691

(22) Filed: Aug. 18, 2015

(51) Int. Cl. A61B 5/00; A61B 5/11; A61B 5/107

(52) U.S. Cl. CPC A61B 5/0022; A61B 5/1071; A61B 5/11; A61B 5/6801; A61B 2562/0219; A61B 2503/10; H04W 84/12

(57) ABSTRACT

Systems and methods for systems and platforms that receive data from and send data to wearable devices, receive data from environmental sensors, and visualization which collectively creates a kinematic chain to allow for input from individuals, groups, third party information and interfaces with historical data as well as predictive modeling systems. Systems that include data from interoperable wearables in conjunction with one or more sensors and measuring devices may be used. Furthermore, the interoperability of one or more sensors and communication modules could be networked with one or more physical entities and objects creating an interactive communication network. Wearable platforms could include but are not limited to all wearables (inter- and intra- and proximity-wearables), smart devices, IT networks, virtual systems and secure information processes. In addition, the platforms and networks could also include real-time biological interoperability based on various synchronized and/or asynchronized data inputs, information and media.

### BACKGROUND

This application is an utility titled “Interoperable Wearable Devices and Communication Platform”, and the teachings of which are incorporated herein in their entireties by reference. This application claims the benefit of the filing date of U.S. provisional application filed on date Jun. 29, 2014.

The present application relates to a plurality of wearables, biosensors, visional kinematics and integrated analytics which accurately measures and analyzes movement or various metabolic and motion activities individually or as a group collective in any sports environment or scale.

In the present disclosure The use of sensor technologies has been forecasted to meteorically increase as wearables become more advanced and interactive. Environmental, biological, scientific and other data can currently be measured and transferred to various computer-based or smart devices. However, synchronized and/or asynchronized information requires greater interoperability between various types of wearable devices as it relates to one or more inter-nearables and intra-wearables, proximity-wearables, biosensor enabled wearables, and kinematics, statistics, computational geometry, optimization etc. when creating an integrated and multilayered and multifunctional group of intersecting wearable measures referred to in the present invention as a Players Wearable Platform (PWP).

Presently, wearables measure relatively singular task and minor tasks such as a person body temperature, heart rate or oxygen level when involved in fitness working outs in order to calculate calories burns, track steps, distance, and stairs climbed and sleeping pattern. It's only synchronized to individual computer or selected smart phone. It is not cohesively integrated nor designed to include complex tasks and an interoperable measurements as a team or unit of team or group.

Biosensor could be defined in the present invention as an analytical device, used for the detection of an analytes that combines a biological component with a physicochemical detector including but not limited to:
the sensitive biological element (e.g. tissue, microorganisms, organelles, cell receptors, enzymes, antibodies, nucleic acids, etc.), a biologically derived material or biomimetic component that interacts, binds, measures, recognizes and identifies. ,
the transducer or the detector element(s)-physicochemical; optical, piezoelectric; electrochemical, etc. that transforms the signal resulting from the interaction of the analyte with the biological element into another signal (i.e., transduces) that can be more easily measured and quantified;
biosensor reader device with the associated electronics or signal processors that are primarily responsible for the display of the results and measurements.

Intra-wearables are defined as objects inserted inside a body and/or under/through the layer of skin or animal's coat or underneath the animal's coat. Proximity-wearables are defined as objects which are neither contacting the epidermis nor the integumentary system. Furthermore, proximity and another embodiment could be defined as “close to”, “near”, within geographic vicinity to a person or animal.

Kinematic chain modeling and image tracking can be defined as a branch of classical mechanics which describes the motion of points, bodies (objects) and systems of bodies (groups of objects) without consideration of the causes of motion. The study of kinematics is often referred to as the “geometry of motion. Kinematic analysis is the process of measuring the kinematic quantities used to describe motion.

In addition, kinematics motion could be defined as one or more trajectories of points, lines and other geometric objects and their differential properties such as velocity and acceleration of the human body.

The study of kinematics can be abstracted into purely mathematical functions. For instance, rotation can be represented by elements of the unit circle in the complex plane. Other planar algebras are used to represent the shear mapping of classical motion in absolute time and space and time. Kinematics parameter in geometry, mathematicians, geometric transformations and the movement of components of a mechanical system simplifies the derivation of its equations of motion, and is central to dynamic analysis.

Sensory inputs such as visual (sight), touch, auditory, olfactory and taste which could be interoperably measured by one or more biosensors.

Multiple computerized inputs (enriched data) could be defined as any multimedia technology which collects and enriched data such as but not limited to high definition cameras (video and multi-frame pictures), mobile cameras, auditory microphones, speakers, vibration, wearable trackers, biosensors etc. These inputs could be derived by one or more persons, one or more geographies and from one or more functions and purposes.

It is understood by anyone familiar with the art that all data inputs could be analyzed, stored, reproduced in any scale, any 2D or 3D measurement, any mode, any algorithm, any geometric relationship, any method, any linearized flow model, any intersecting reconstruction, any encoding and decoding, and keypoint(s), any preprogrammed parameter, any time analysis, any probe from covariance analysis, any form of human tracking, any source, any protocol any format, any database, any server, any network and scaling, any sport, any team organized training, any correspondence, any wearable and other inputs and any processor etc.

In addition, Motion perception could be defined by many disciplines, including psychology, visual perception, neurology, neurophysiology, engineering, and computer science however, the combined measurements of multiple measurements derived by multiple wearables on one or more individuals currently do not meet the standards required when interchanging a multitude of wearable units used for example sports and team performance and training.

In addition, Inter-wearables are defined as objects occurring in where it meant “between,” “among,” “in the midst of,” “mutually,” “reciprocally,” “together,” “during”, etc. In addition, inter-wearables are defined as any wearable, smart device or measuring device which contacts the skin (epidermis—stratum basale, stratum spinosum, stratum granulosum, stratum licidum, stratum corneum) and any integumentary system which includes all contact with a body for measurement.

Currently, wearable inefficiency results from a lack of a systematic and integrated informational system. The current system is largely based on “individual measures”, and doesn't distinguish between individually derived wearable data when used as an integrated component of enhancing a team's overall performance. In addition the current system lacks the ability to integrate one or more of the following:
Individual wearable data used as a team component from as a plurality wearable “inter” and “intra” devices.
Team data derived by a composite of wearable devices data based on individual performance.
Measurable data on groups or sub-groups of one team.
Measurable data on groups or sub-groups of an opposing team.
Adjustment of individual performance based on wearable data analysis.
Adjustment of group or subgroup performance based on personal and team wearable data analysis.

The present invention relates to an interchangeable functionality of one or more wearable devices. An example of one embodiment of visual and ocular inputs and interpretations could be interconnected through one or more networks and/or platforms. Individual wearable data used as a team component from as a plurality wearable “inter” and “intra” devices. Accordingly, the Players Wearable Platform and Communication (PWPC) system, methods and devices related to and used in conjunction therewith are provided which address the needs and provide the advantages outlined herein.

### SUMMARY

Player Wearable Platform (PWP) and Players Wearable Platform and Communication (PWPC) system could be defined by one embodiment as the ability to detect data from one or more wearable devices, data from one or more Player Wearable Networks with intra or inter proximity devices. Furthermore, these wireless devices could include but not limited to all deviations of biosensors capability and functions, all wireless servers and networks, and data analysis. The above could include data transfer and retrieval from all smart devices compatible with the PWP system. PWP devices not limited to sensors attached to the body, in the body or near the body, smart wearables including but not limited to; smart clothing, mouth-guards, smart hats, smart helmets, smart mouth guards, smart knee supports, smart (ankle supports, arm supports, leg supports), smart socks, smart tracking (balls, pucks, bats, hoops, baseboards, sidings, hockey sticks, lacrosse sticks etc.), goggles, smart shoes, etc. It is understood by anyone familiar with the art that inter- or intra-wearable technologies, RFID tags, all drone technology, near field communication devices, infrared, facial recognition system, motion detectors, high definition video, M2M, location based such as indoor/outdoor positioning (XY) (XYZ)-GPS, as defined in the PWP embodiment(s), high definition video and imagine overlay technology. The PWP system can be used to monitor vital signs of an athletic, comprising: and wearable sensor array comprising at least on sensor elements situated on the player, the at least two sensor elements including an accelerometer and a non-accelerometer sensor, the accelerometer configured to measure at least one accelerometer-measured bioparameter of the athlete from among: resting patterns, activity patterns, movement patterns, position patterns, and the non-accelerometer sensor configured to measure at least one of the following non-accelerometer-measured bioparameters of the athlete such as but not limited to: temperature, pulse rate, respiration rate, oxygen (o2) sensors, dehydration; one or more remote or local processors configured to receive (a) sensor output data from the sensor array concerning the measured bioparameters and (b) reference data concerning the measured bioparameters of the athlete team including the one or more remote or local processors configured to determine whether a specific medical condition is suspected by at least one of the following:(i) recording at least two bioparameters relative to the reference data and comparing a cumulative score of all recorded bioparameters to a threshold cumulative score or to a threshold cumulative range; or (ii) identifying an abnormal pattern in at least one bioparameter from among the accelerometer-measured bioparameters and the non-accelerometer-measured bioparameters, the one or more remote or local processors configured to send an alert if at least one specific medical condition is suspected.

PWP team data derived by a composite of wearable devices data based on individual performance. Kinematic analysis may be used to find the range of movement for a given mechanism, and, working in reverse, kinematic synthesis designs a mechanism for a desired range of motion. In addition, kinematics applies algebraic geometry to the study of the mechanical advantage of a mechanical system or mechanism.

Detection and discrimination of motion can be improved by training with long-term results. Participants trained to detect the movements of dots on a screen in only one direction become particularly good at detecting small movements in the directions around that in which they have been trained. However perceptual learning is highly specific. For example, the participants show no improvement when tested around other motion directions, or for other sorts of stimuli.
Measurable data on groups or sub-groups of one team.
Measurable data on groups or sub-groups of an opposing team.
Adjustment of individual performance based on wearable data analysis.
Adjustment of group or subgroup performance based on personal and team wearable data analysis.

In one embodiment of the current invention, the PWP system can integrate the motion process as previously described as an integrated map for athletics and coaches to help understand and modify, a player's teams performance as individuals and as a collective. The collective gathering of data inputs, vestibular and proprioceptive inputs for example could create a collective performance map for athletics in any sport and team organization. Perceptual learning of motion could be used for athletics and coaches to build good cognitive and muscular habits and to eliminate bad habits.

In another embodiment PWP integrates visual motion detected with cameras, biosensors detection and spatio-temporal correlations between players. The PWP processing smart system generates plausible models for one player or a group of players.

In addition, PWP could provide a means of coherence assessed by measuring the ratio any geometric threshold which lifts a 2D representation of a body to a 3D, the visual input will be a 2D projection of a 3D scene. The motion cues present in the 2D projection will by default be insufficient to reconstruct the motion present in the 3D scene. Put differently, many 3D scenes will be compatible with a single 2D projection.

In addition in another embodiment of the present invention the PWP software could separate or control in inclusion or exclusion of extraneous objects for example the building and structure where the game is being played, fences, fans and other factors which interfere which any analytic inputs of motion and other stimulus as previously described.

PWP also could provide artificial intelligence models which could augment a cognitive map which serves an individual to acquire, code, store, recall, and decode information the relative locations and attributes of phenomena in their spatial environment for one or more players.

PWP could make available the ability to integrate numerous team and individual sports models as exemplified by the following models:
Individual wearable data used as a team component from as a plurality wearable “inter” and “intra” devices. For example, San Antonio Spurs players wearing smart trackable wearables (mouth-guards, arm braces, smart sock and clothing) video and biosensors. When practicing the basketball guard could bring a smart-ball which has sensors down the court, he passes the ball to the forward opposing player misses his/her change to block the pass. The PWP system could provide the coach with real-time or near time information to reposition the guard to help his or her defense. In addition, the PWP system could help train the guard by giving direction inputs such as but not limited to vibration to directionalize a player's movement. For example the player's wearables could vibrate on the right hand wearable support to signal the players shift to the right. It is understood by anyone familiar with the art that the PWP system could provide the player with any form of direction or movement (height, right, left, front, back etc.). In addition it is understood by anyone familiar with the art that the PWP system could alert spacial proximity with other players, motion correction when passing the ball or making a basketball shot and other inputs provided by the coach for example. If a player misses the ball the player network of PWP wearables on the player's body could help correct the movement by vibration or other methods. In addition, the basketball itself could be smart and trackable (traceable), as well as the hoop and baseboard. Smart wearables and biosensors worn by the player coupled with a smart chip imbedded in the ball as well as sensors on the baseboard, rim and net could interoperate in a cohesive system when athletes are training or in a regular game situation. It is understood by anyone familiar with the art that hi-definition cameras takes and other data could also be incorporated integrated with wearables and biosensors in the PWP and PWPC system. The PWP comprehensive data input integration could help coaches, players, staff, and teams more efficient. It is also understood by anyone familiar with the art that all sports, all organizational or governmental, military teams could profoundly learn from the PWP system and performance enhancement software which could help correct movement, position and overall performance.
Team data derived by a composite of wearable devices data based on individual performance. The PWP system could also provide one or more biometrics in order to determine a player's O2, hydration, pulse, temperature individually or as a collective. For example the players mouth-guard could contain biosensors which through the oral cavity could determine a player's energy level and when he needs to be replaced. The PWP system could also account for external factors such as but not limited to altitude, the opposing teams courts and fans, noise levels and other related external factors.
   Measurable data on groups or sub-groups of one team. The PWP system could also provide the coach or manager a composite map of how each player's performance could be enhanced and optimized by resting a certain time through the oral biosensor mouth-guard. It is understood by anyone familiar with the art that all sports, motions, biosensor data and visual components could be integrated through the PWP as previously described.
   Measurable data on groups or sub-groups of an opposing team. The PWP system could provide information of the opposing team or group if authorized.
   Adjustment of individual performance based on wearable data analysis. The couch could correct bad habits through the PWP system. When a player throws from the free throw and misses for example could be corrected when in practice by the coach through the player's wearable network as previously described or through an ear piece worn by the player.
   Adjustment of group or subgroup performance based on personal and team wearable data analysis. The PWP system could also provide comprehensive database and historical analysis. The PWP system could be exemplified by a basketball player named James. James Jersey No. 1 is wearing smart devices when training for his basketball team. His team makes are also wearing the smart PWP devices and have split the squad in half for a practice scrimmage. The PWP measures bioparameters for all 10 players on the court. The PWP database is concurrently in real-time or near-time analyzing movement efficiency with individual and team performance. The PWP system will compare each player's current performance with their historical database, historical performance, biomarkers and bioparameters.

Accordingly, the Players Wearable Platform and Communication (PWPC) system, methods and devices related to and used in conjunction therewith are provided which address the needs and provide the advantages outlined herein.

Medical and physiological device inputs examples:
Temperature, Pulse and heart rate Fatigue Respiration;
Pictures of skin with scale at different times (changes in size or color of spots can be compared over several days, weeks, months, and years);
Blood oxygen level CBC;
Anything that changes pH of body fluids—saliva, sputum, epithelium from mouth, nose, anus, vagina, or urine or a drop of blood;
Blood sugar, enzymes, hormones, chemicals, blood chemistry, or other still not known chemical components in a drop of saliva, sputum, epithelium from mouth, nose, anus, vagina, urine or a drop of blood;
Eye exams, nose exams, ear exam;
Balance and agility test;
Sonograms to see inside for heart function, pregnancy, blood vessel function and thickening, lung function, kidney function;
Lung function with blowing into a tube. Stool test results
Hair follicle contains enough DNA for some test. body fat amount, muscle amount
Skin biopsy
X-rays, MRI, CT scan and other imaging tests EKG
Dehydration level Perspiration
Pain level Vision
Muscle and Tissue condition
Sleep Nutrition
Height weight Injury Illness
Pharmaceutical utilization including Prescription and over-the-counter medications and injections Vitamins, Supplements
Performance enhancing medications or therapies
Fitness and Wellness, such as but no limit to monitoring muscle soreness, injury recovery, sleeping pattern; tracks steps, distance, calories burned and stairs climbed; animate performance and team coordination.

Infotainment functions, such as but no limit to,
Group dancing, cheer-leading, training, etc.
Industrial and Corporate uses, such as but not limit to worker productivity, company security, warehouse inventory management, etc.
Education, such as monitoring of students' learning progress and learning style Sports such as monitoring players' performance and training players Entertainment such as gauging audience reaction
Military such as tracking the location and health status of troops on the ground
Research such as studies of the physiological data and mental status/condition data of individual and animals under many types of conditions

Citizens can have type of sensors interconnected with other people who shared commonality, including but no limit to an event or multiple events, exemplified by the following:
Sport events or practices interconnected required team effort
Public experiences with intersect and/or interact with each other
Public events required commonality and/or cohesive movements or tasks coordination
Educational interoperability to improve learning and behaviors Corporation and government training.

Specifically, PWP could include a series of events resulting from the detection or the crossing of personal data collection, and provide direction as needed. One scenario could include for example, the following: Sport training, the wearable device could collect data and analyze performance information through software settings including but limited to:
How is environmental factors effect sport performance, such as Environmental conditions present at the time of the sensor readings including but not limited to heat, humidity, precipitation, lighting, winds, storms;
Enabling or disabling the ability to imitate, animate and measure performance in order to enhance and improve future ability;
Enabling or disabling real-time movement analysis, either by manual personalized coaching or by pre-programmed M2M computer processing;
Query certain information regarding one or more specific factors and/or variables which could improve performance and efficiency;
Individual and team coordination training for any type of events, such as but not limit to sporting events, emergency events, corporation events, transportation and public events, etc.
Be able to put collected data into a central database or secured database for public and/or private team and/or government/organization to evaluate;
Predictive modeling based on an individual's play, team play and hypothetical individual or team play Ability to integrate historical play information with current players to compare individual and team performance including modeling performance of a batter against a particular pitcher or an athlete against another athlete current, historical or hypothetical performance.
Differentiate and distinguish one team player from another utilizing facial recognition and imaging, body recognition, biological recognition and other methods of PWP software processes.

Data from a wearable device or data from a Personal Wearable Platform or Personal Wearable Network could be combined with data from one or more of the following:
Individual data from wearable device or devices
Group data from wearable devices Self-reported data on an individual Self-reported data on groups or sub-groups
Third Party data on individuals and or groups or sub-groups
Environmental Sensor data on living and non-living objects Weather Data
Location Data, Lighting Data, Audible Data, Visibility Data, Sensory Data, Olfactory Data, Taste Data
Historical Information Reference data Scheduling information, Cost information, Supply information, Availability information, Forecasted information, Modeling programs, Treatment Options Medical Data
ERP Systems Data: Quality Data Profile Databases Testing Systems Survey Systems; Capacity and Planning Systems
Travel Systems Media Data Options Data Research Data
   wearable tech devices includes data from the primary person or animal being measured and data from other people or animals in proximity to the primary person or animal. Information and Data can be received and/or transmitted creating a bi-directional flow of data in real-time, near time or delayed time.

Environmental Sensor data on living and non-living objects:
Weather Data, Location Data, Lighting Data, Audible Data, Visibility Data, Sensory Data, Olfactory Data, biological Data, Test Data;
Historical Information, Reference data, Scheduling information, Cost information, Supply information, Availability information, Forecasted information, Modeling programs Treatment Options, Medical Data;

The PWA system could be applied and may be part of a plurality of dental devices including, but not limited to, caps, crowns, bridges, mouth-guards, denture, implants, veneers, fillings, fixed prosthesis, braces, and/or wires, and retainers, mouth-guard, occlusal splints and/or temporary/removable materials, used in dentistry and recreationally (tongue piercing, etc.). It is understood anyone familiar with the art that a plurality of biosensors and RFID components could be installed in any dental device and/or placed in any oral cavity depending upon the medical or diagnostic intent. In yet another embodiment, the PWA systems could be used in conjunction with other smart wearables on one or more individuals creating a measurable team diagnosis. Furthermore, smart wearables or other medical devices could utilized by a collective group including any team sport application i.e., basketball, soccer, baseball, hockey, swimming, track, football, etc. It is understood that the PWA system could measure and diagnose individual performances in sports (singularly measured, analyzed and diagnosed) or measured, analyzed and diagnosed collectively as a team composed by individual players.

The PWP will provide a method to centralize data collection, exemplified by the following: Personal medical, physical data and health care, such as but not limit to hearing aids, ECG monitors, PERS patches, Smart glasses and contacts (both prescription or non-prescription), blood pressure monitor, medical alert, continuous glucose monitoring, diabetes monitoring, defibrillators, prescription or non-prescription pharmaceutical delivery product, dehydration, pulse oximetry and other medical health care and physical data/measurement and alertness. Examples of medical data inputs may include but are not limited to:

Infotainment functions, such as but no limit to, all team sports, group dancing, cheer-leading, training, etc.

Education, such as monitoring of students' learning progress and learning style Sports such as monitoring players' performance and training players Entertainment such as gauging audience reaction. Military such as tracking the location and health status of troops on the ground. Research such as studies of the physiological data and mental status/condition data of individual and animals under many types of conditions.

Citizens can have type of sensors interconnected with other people who shared commonality, including but no limit to an event or multiple events, exemplified by the following:
Sport events or practices interconnected required team effort
Public experiences with intersect and/or interact with each other
Public events required commonality and/or cohesive movements or tasks coordination
Educational interoperability to improve learning and behaviors Corporation and government training.

Specifically, PWP could include a series of events resulting from the detection or the crossing of personal data collection, and provide direction as needed. One scenario could include for example, the following: Sport training, the wearable device could collect data and analyze performance information through software settings including but limited to:
How is environmental factors effect sport performance, such as Environmental conditions present at the time of the sensor readings including but not limited to heat, humidity, precipitation, lighting, winds, storms;
Enabling or disabling the ability to imitate, animate and measure performance in order to enhance and improve future ability;
Enabling or disabling real-time movement analysis, either by manual personalized coaching or by pre-programmed M2M computer processing;
Query certain information regarding one or more specific factors and/or variables which could improve performance and efficiency;
Individual and team coordination training for any type of events, such as but not limit to sporting events, emergency events, corporation events, transportation and public events, etc.
Be able to put collected data into a central database or secured database for public and/or private team and/or government/organization to evaluate;
Predictive modeling based on an individual's play, team play and hypothetical individual or team play Ability to integrate historical play information with current players to compare individual and team performance including modeling performance of a batter against a particular pitcher or an athlete against another athlete current, historical or hypothetical performance.

The system could accommodate sports related data such as but is not limited to: Results of medical treatment such as surgery, therapy, or injections; Any type of therapeutic procedures, Other test results prescribed by a physician or undertaken by the individual to assess or improve the physical and/or mental functioning, awareness, knowledge to enhance performance.

Performance Measurements such as but not limited to: speed, acceleration, jumping, field, course and court information such as the type of turf, floor, moisture level of greens, etc.

Biometric Performance measurements could be individually, dehydration and electrolyte balance (oral): Sodium normal levels 135 and 145 mEq/liter (135-145 mmol/L), normal serum range for chloride is 97 to 107 mEq/L, normal blood potassium level is 3.5-5.0 milliEquivalents/liter (mEq/L), or in international units, 3.5-5.0 millimoles/liter (mmol/L). Sodium and Chloride are most important and blood volume can be calculated based on their blood concentrations.

Heart rate Pulse and Oximary or blood oxygen levels: 60 to 100 beats a minutes but for athletes it might be as low as 40 beats/minute; In medicine, oxygen saturation (SO₂), commonly referred to as “sats”, measures the percentage of hemoglobin binding sites in the bloodstream occupied by oxygen. At low partial pressures of oxygen, most hemoglobin is deoxygenated. At around 90% (the value varies according to the clinical context) oxygen saturation increases according to an oxygen-hemoglobin dissociation curve and approaches 100% at partial oxygen pressures of >10 kPa. A pulse oximeter relies on the light absorption characteristics of saturated hemoglobin to give an indication of oxygen saturation. Temperature: 97.8 degrees F. (or Fahrenheit, equivalent to 36.5 degrees C., or Celsius) to 99 degrees F. (37.2 degrees C.) for a healthy adult. Respiration rates: 12 to 16 breaths per minute. EKG- Blood pressure: 120/80 of Hg and athletes may have lower.

EMG or Electromyography to evaluate muscle activity inertia sensor.

The system can maintain real time, near-time and/or historical data. It could analyze conditions such as when lactic acid builds up in the muscles. The system can model individual and team behavior/athletic performance, and the prospective data such as anticipated ability upon return from injury.

The system allows for players' information to be loaded into the individual training and group programs which model individual and group/team play. The information allows for the virtual scouting database which allows for a player's profile including all the historical data and predictive data to be stored, retrieved, modified, imported and exported.

The system can model play based on changes in physical attributes such as strength training, flexibility or surgery. Historical team play can be reenacted through the system as well as projected team play. Integrate data from and interact with smart equipment including but not limited to balls, bats, goals, bases, nets, sticks, helmets, lines, proximity lines, pucks, etc. Referee's data could be integrated into the system for more accurate status of the game and historical reference.

Industrial and Commercial Interoperable wearable devices and network in a commercial industrial setting: the employees, consumers, customers could all provide input into the system. In a manufacturing example the employees could be wearing wearable sensors which could measure productivity, accuracy, quality and other physical and mental inputs. Passengers could be more easily scanned and their luggage data could be associated with the passenger. Passengers traveling on the plane could determine the health status of the person sitting next to them and react accordingly.

Data from a wearable device or data from a Personal Wearable Platform or Personal Wearable Network could be combined with data from one or more of the following:
Individual data from wearable device or devices
Group data from wearable devices Self-reported data on an individual Self-reported data on groups or sub-groups
Third Party data on individuals and or groups or sub-groups
Environmental Sensor data on living and non-living objects Weather Data
Location Data, Lighting Data, Audible Data, Visibility Data, Sensory Data, Olfactory Data, Taste Data
Historical Information Reference data Scheduling information, Cost information, Supply information, Availability information, Forecasted information, Modeling programs, Treatment Options Medical Data
ERP Systems Data: Quality Data Profile Databases Testing Systems Survey Systems; Capacity and Planning Systems
Travel Systems Media Data Options Data Research Data

To create a system with which to but not limited by receive, retrieve, analyze, predict, modify, transmit, share, secure and store information. The system accommodates data files such as but not limited to audio files, video files, image files, 3-D representations, holographs, live feeds from the network, live feeds from the internet and information files.

### DETAIL DESCRIPTION

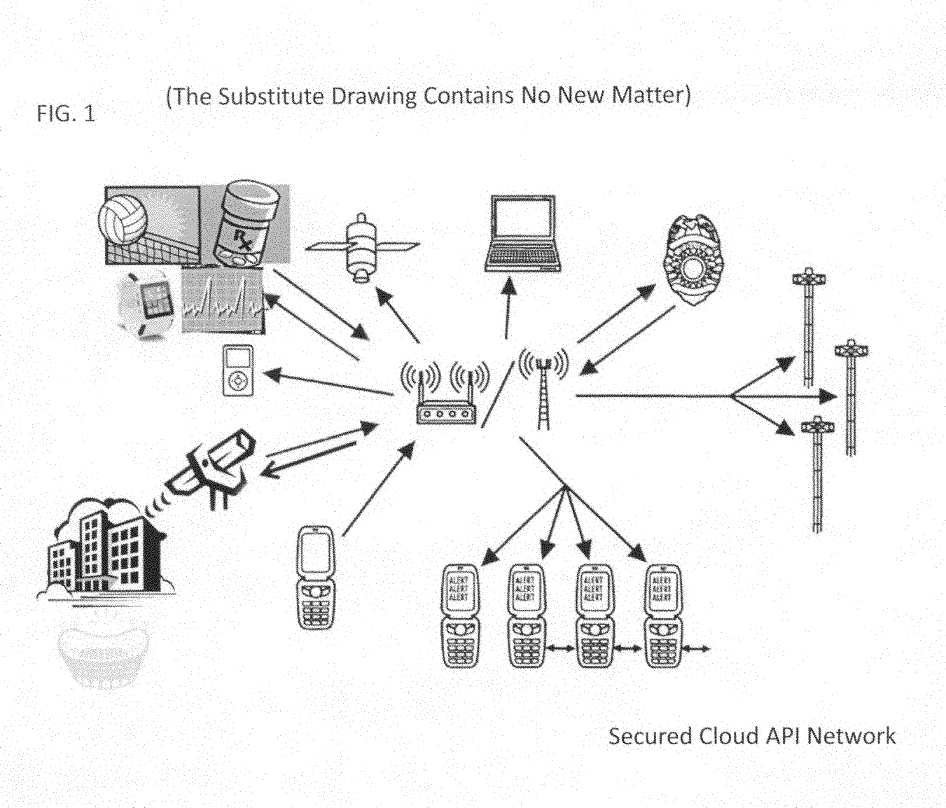

FIG. 1 shows a block diagram of one or more of mobile devices and/or wearable devices creating virtual access and retrieval secured system network for a plurality of various locations and devices, determining one or more geographic locations, and one or more devices communicating together as a team/group or as an individual form, in accordance with embodiments of the present invention. Mobile devices and/or wearable devices can receive data in a predetermined physical position and movements, such as but not limit to: player's speed, biological information, physiological information, proximity of players in relationship to other players or teams, such as but no limit to, base, bats, goals, sport fields, demarcation, watersports, angle of movements, mapping (2D, 3D, holographic, pictures, video or sequential events for all media types and protocols) for location based sport information, activities and events. Various data could be analyzed in order to increase individual and/or team's performance. In addition, the system could collect real-time or near-time or delay time information such as but not limited to: physical experience, team activities, individual's behavior and other data. Each mobile device is capable of seamlessly interfacing customized protocols for school campuses, corporate complexes, industrial centers, governmental buildings, retail developments, cities, municipalities, sports stadiums and other organizations or properties (generally, a “campus”) to facility group activities, training and session, etc.

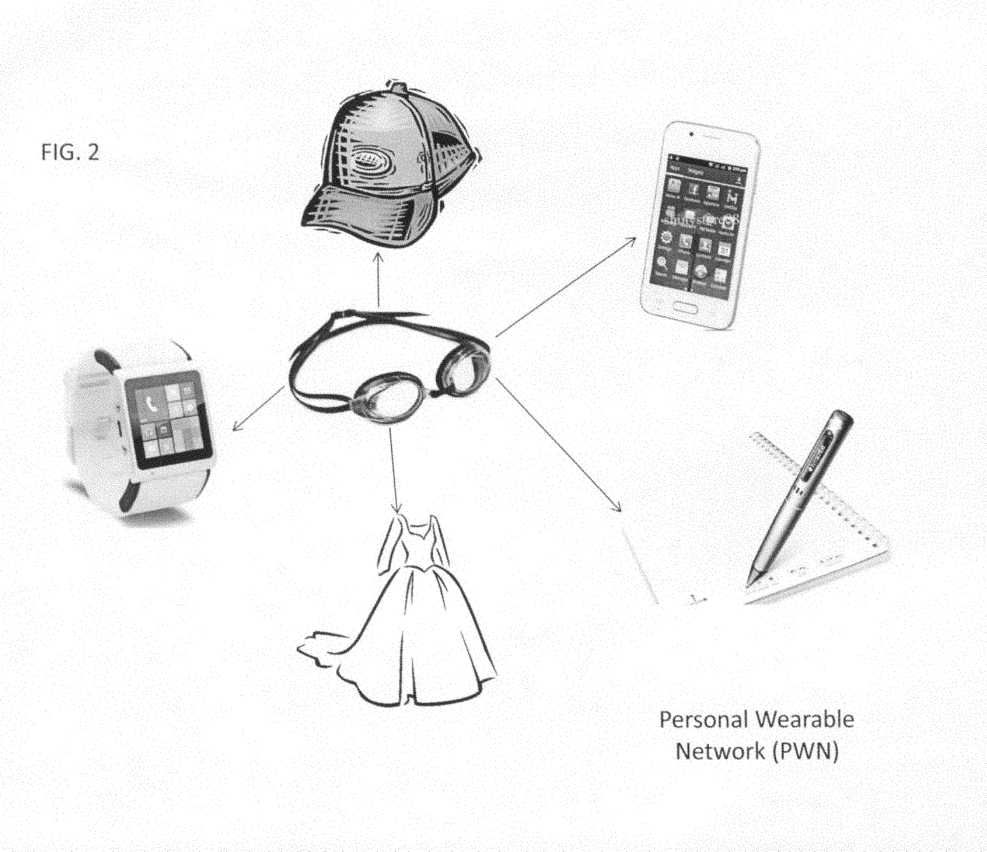

FIG. 2 shows a representation exemplified a network of wearables which could create one or more unified functions and applications based on one or more smart devices, such as but not limit smart phones and tablets, smart clothing, and smart hats, smart jewelry, smart bands (hand bands, wrist bands, leg bands), neckwear, finger-wear, shoes, boots, gloves, and eyewear, contact lens, glasses and all body-wears, belt, tooth protection or month guard, etc. In addition to inter-wearable, intra-wearable could be inserted into body to measure all medical applications. Furthermore, wearable device could be tethered or in geographic proximity to one or more users. In addition wearables, whether be inter-, intra- or proximity, could performance one function or multiple functions and tasks in one or more collective network. These tasks could be customized based on the need of one or more users could be connected to any cellular network or wireless or wired line communication, satellite communication and all means of connectivity networks, information database and communication networks. Communication could be interoperated to perform a scheduled real-time or near real-time task and/or application. The independent wearables or network of multiple wearables could be independently processing and sending information to one or more networks, or could be integrated to any mobile platform as well as any computerized system, and could receive and transmit information independently or integrated. Another example could be based on real-time data, feedback and analysis of the information gathered by one or more wearables in order to change behavior or direction to enhance performance, life style, motion and activities, in accordance with embodiments of the present invention.

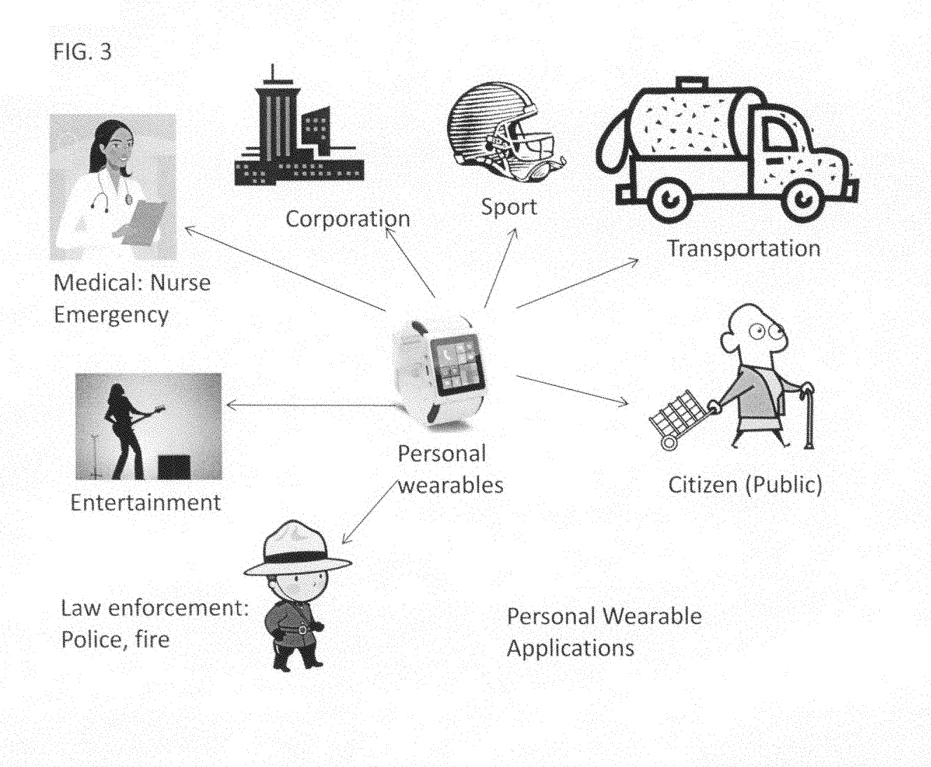

FIG. 3 is an example of one or more wearables been integrated to various applications, such as but not limit to medical, law enforcement, transportation, corporation, and general public sector. It could be integrated within the sector and/or cross-functionalized to multiple sectors, in accordance with embodiments of the present invention.

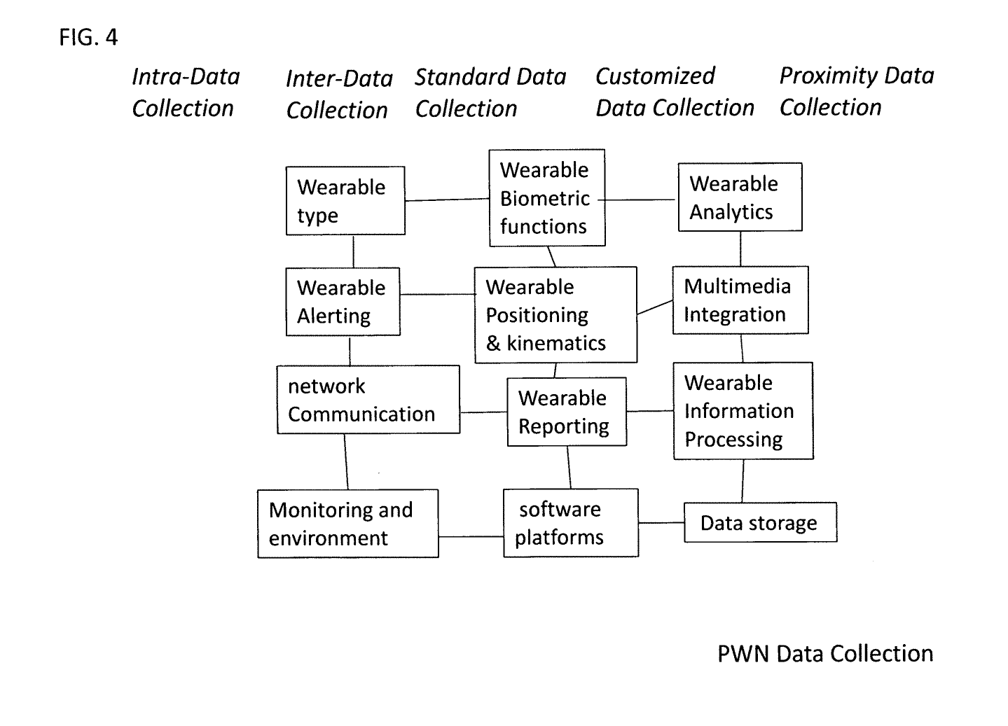

FIG. 4 shows an example of a system employing one or more of the wearable devices and systems and functions in order to access and retrieve information from a plurality of various wireless access and device to detect and track user's positions (indoor/outdoor positioning and GPS) based on individual customizable needs and correlating information from wearable networking and retrieval personal and/or social network information and data, in accordance with embodiments of the present invention. Wearable devices can send and/or receive notifications in a predetermined data series for individual or social based information and events. Various wearables could be customized in order to meet required functionalities and needs. In addition, the system could collect real-time or near-time or delay-time information.

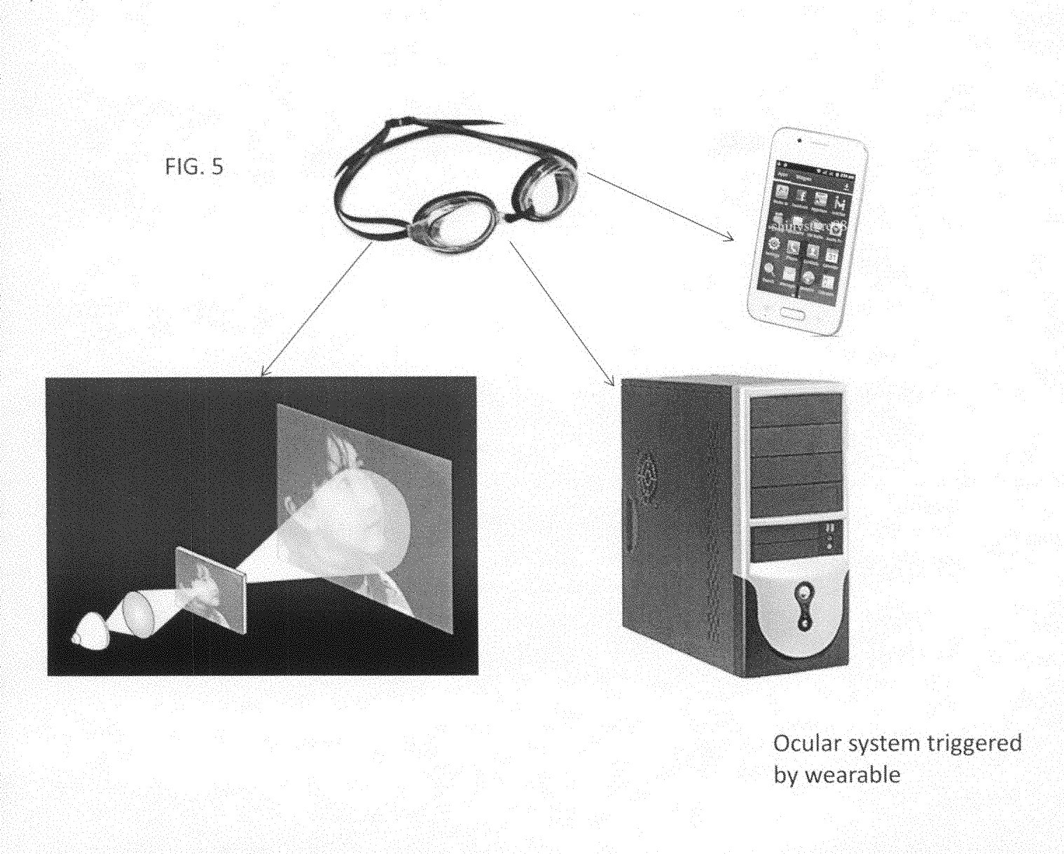

FIG. 5 is an example of a smart ocular system becoming an interactive and individual screen display through a computer chip based on the lens itself to display visual, multi-media, 2D/3D, hologram and audio, and/or a projector built in the eyewear frame can be projected on the back of one or two eyewear lenses, and create various types of display screen, such as but not limit to LED or OLED screen or a micro-display that only the glasses owner can see it. If the smart glasses are networked with other smart devices, network and/or cloud, it can provide presentation or other information in relationship to other users to have same devices. The information can be stored and/or screened and/or packed.

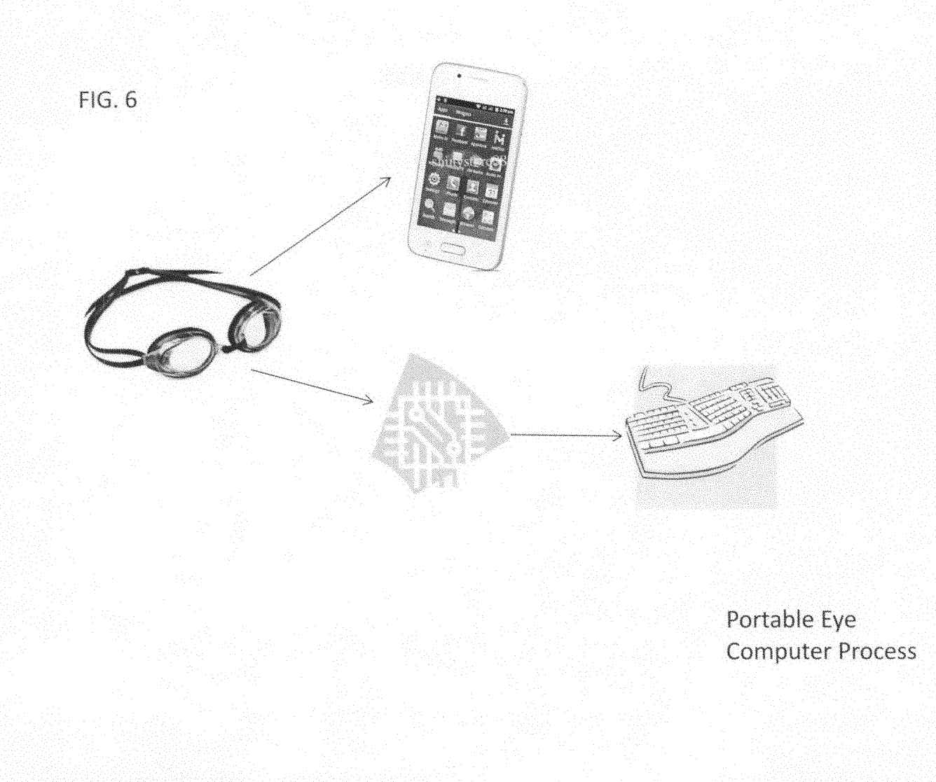

FIG. 6 is an example of mechanism of controlling data in these smart glasses could be done in touching frame or voice recognition or eye movement or body movement. The controlling method can be set up through computer processing system, in accordance with embodiments of the present invention.

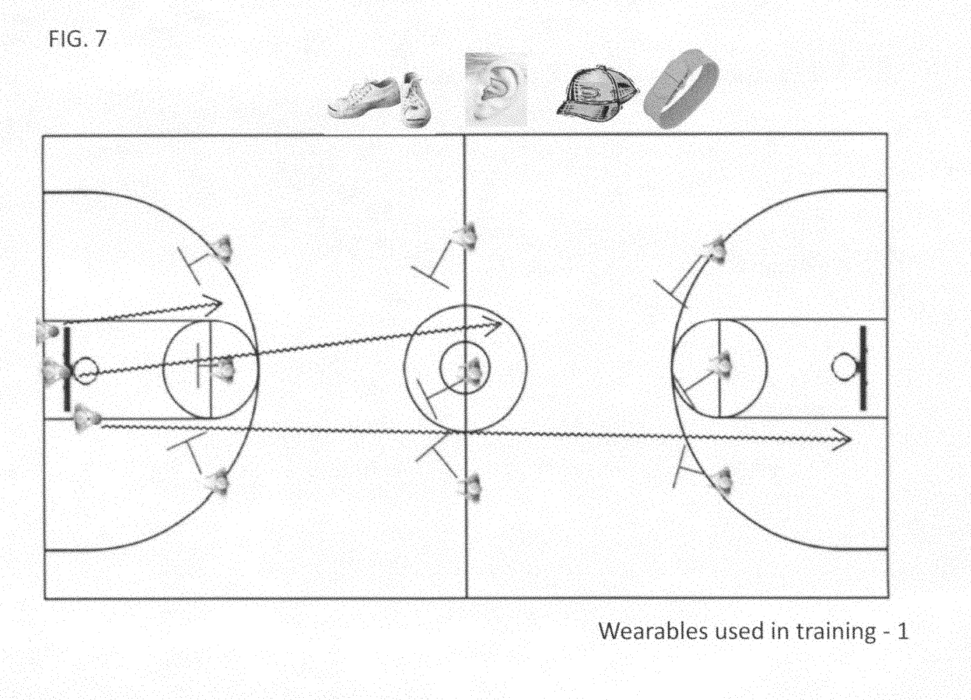

FIG. 7 is an example of a blocked diagram of a basketball court. The representation includes 5 people on each side, practicing and training with coaches and managers. It's understood to anyone knowledgeable of the art, similar variation could be used in all sports, including but not limit to basketball, hockey, football, soccer, golf, track and field, swimming and diving, rugby, lacrosse, field hockey, all water sports, land sports, air sports such as sky diving, when utilizing any functionalities and usage of all wearables and networks. It's also understood that the sports representation could be used in full or part of any and all corporation, military, education, and industrials, government and medical, etc. in accordance with embodiments of the present invention.

In addition, the PWP system could utilize inputs from multiple sources exemplified one or more wearables such as head band, goggles, wrist band, head gear, teeth protector, shoes, kneepad, smart suit, which could be inter- and/or intra- and/or proximity wearables. One or more wearables can communicate position on the court, movements, and team work based on passing the basketball or shooting the baskets. It will not only help the individual player, but also help team to improve performance on the basketball court. In addition, yet another embodiment, bi-directional communication in real-time, near-time or delay-time, could be monitored and accessed by one more coaches or managers in relation to individual play or team play. Information based on one or more wearable data points can be analyzed to correct mistakes during practicing and training, to change position, plays, offense/defense strategy. Smart wearables could sense other players positioning and ball positioning and special relations to enhance performance and training. Smart clothing and wearables could signal and message other players based on vibration, visualization verbally. Furthermore, smart goggles and glasses, etc. can be used to incorporate by manager or coach to either giving direction or changing strategies or change individual or team's current or further behavior, and opponent's strength and weakness. Players could also utilize smart goggles or eyewear in order to receive or transmit information. The multi-media and video can give instruction to one or more players on the field. In addition to basketball, the basketball can have a chip to positioning and monitoring team plays and players. This entire system as described in this example could be viewed in entirety or individually to enhance performance and conditioning. When wearables networked could be analyzed each play, each movement and each component, and individual player faces and bodies can be super-imposed on the computer, for practicing and learning. The movements can also base on inter-, intra- and proximity. It can also be super-imposed on computer processed existing video with wearable data.

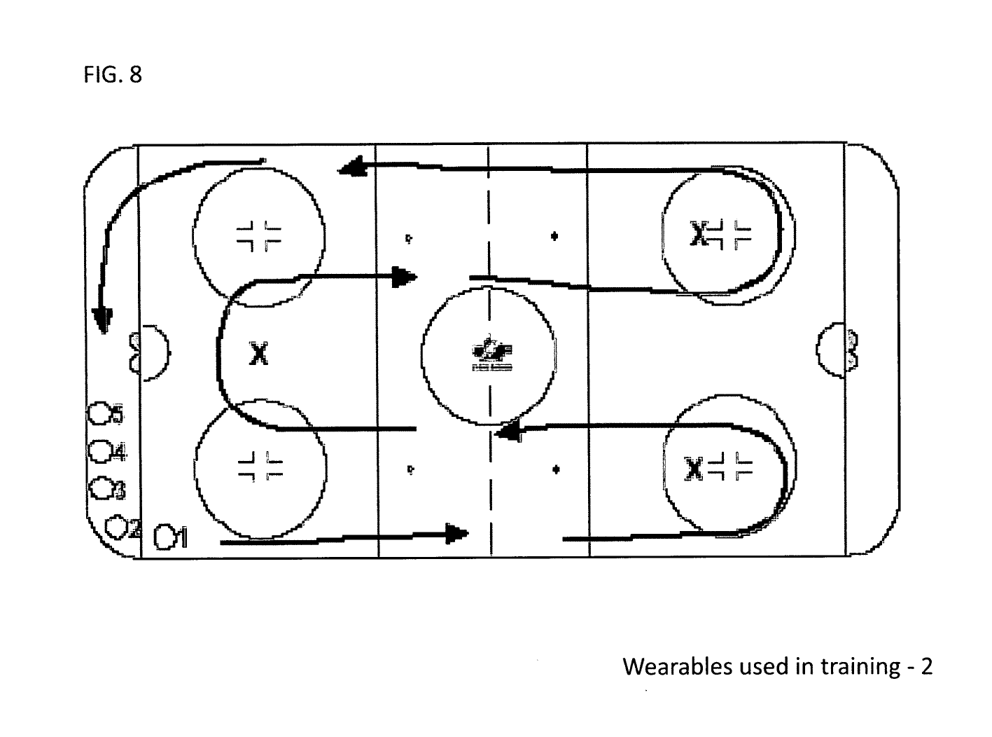

FIG. 8 is an example of computer process which could tracks movements and other information of one or more individuals during individual and team plays. The collected data can also be analyzed and provide bi-directional inputs based on person's and team's performance and activities, and can correct individual and/or team performance in a real-time, near-time or delay-time, in accordance with embodiments of the present invention. Wearable communication devices are interfaced with sport equipment that include one or more embedded sensor to more accurately track when it's been entered playing field zones and goal areas. Wearables could wireland or wireless transmit collected measurements and data from sport equipment to one or more devices and/or network, such as but not limit to accelerometer-based tracking of travel velocity, with room to integrate force-sensitive panels. Collected data could be analyzed through computer process, and display and provide recommendation for best performance through one or more wearables.

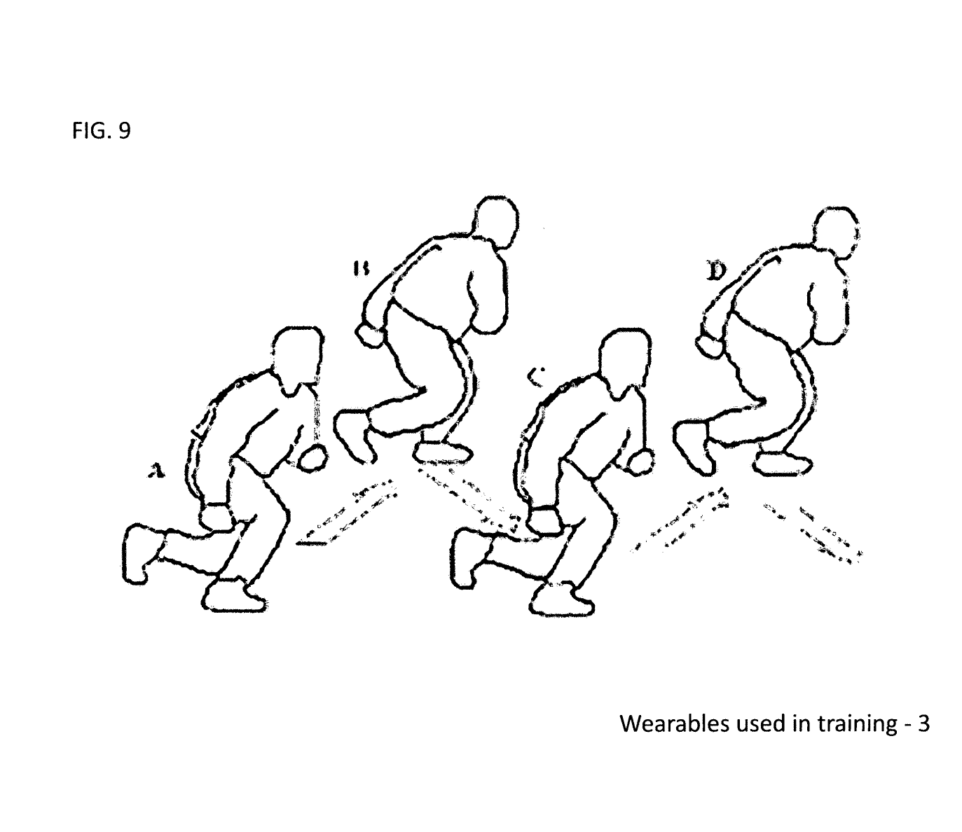

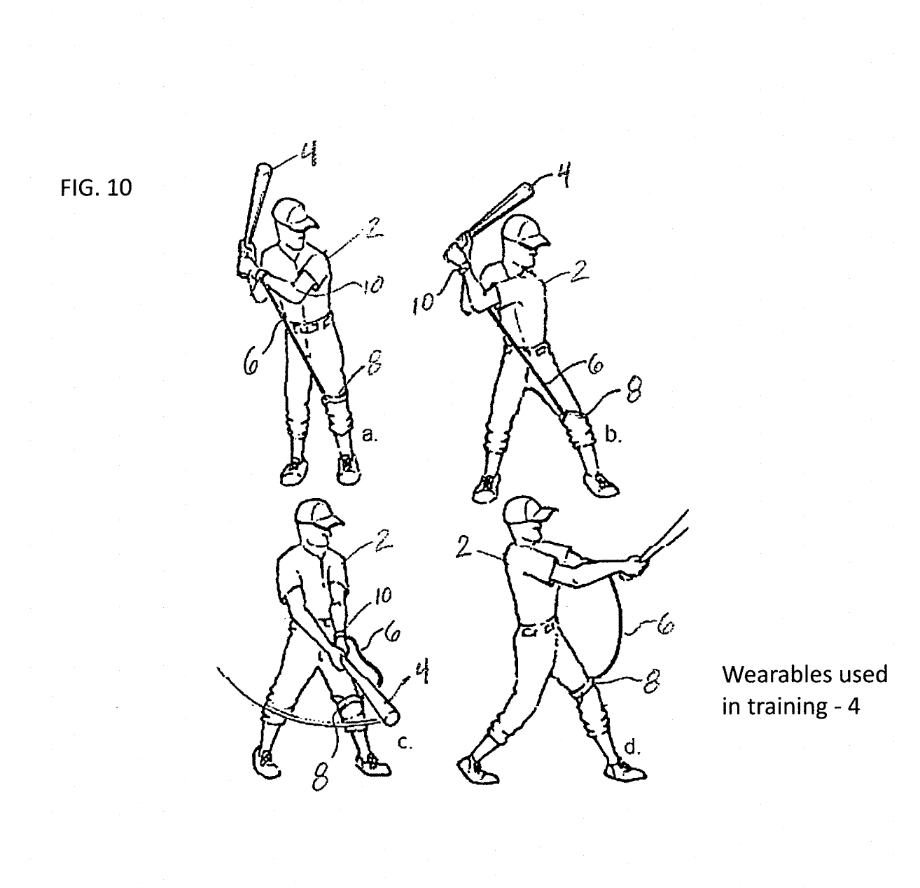

FIG. 9 is an example of how team's cooperating during practice and training to improve their effectiveness. One or more players can use smart wearable devices send data package or data streams, such as but no limit to location, movement, speed, video, audio, biological data, etc. through vibration and/or various kind of messaging systems, in accordance with embodiments of the present invention. FIG. 10 is an example of several wearables worn on each part of baseball batter's body. One or more wearables are interacted and cohesive to each other, and collecting data, such as but not limit to timing, movement, etc. Each wearable can be analyzed individually or jointly due its functionality, and based on data collected by each wearable players can distinguish successful performance, un-successful performance and best fit body movement, to suggest changing of batter's style through analytical computerized approach to improve batter performance and average, in accordance with embodiments of the present invention.

FIG. 10 is an example of using wearable to monitor people's health. One or more sensors are built in wearables to detect parameters, such as but not limit to body temperatures, blood pressure, heart rate and sleeping pattern, etc. Person can set up monitoring range based on personal physical condition and/or physician's advices. Wearable will automatically send out alert to designated individual and/or organization when the data is out of set range, in accordance with embodiments of the present invention.

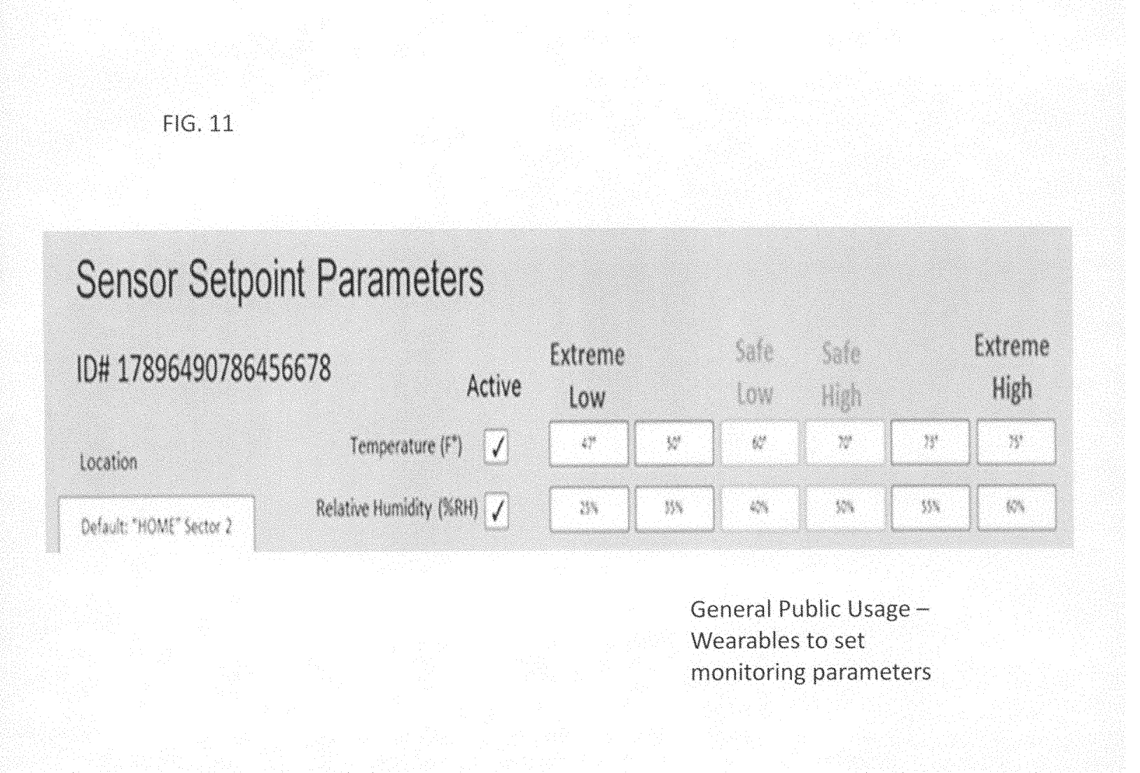

FIG. 11 is an example of using wearables for security monitor and communication, in accordance with embodiments of the present invention. Wearables could connect video monitors through various network, such as but not limit to, Wi-Fi, cellular and satellite, etc. Wearables also have videos recording function itself, and can turn on and transmit to other related parties, such as but not limit to, security guards, administrative personnel, management, etc. For example of using wearables for industrial and warehouse, such as but not limit to, traffic control, inventory management and packing, in accordance with embodiments of the present invention. Industrial and warehouse personnel could use wearables to manage/monitor/communicate daily activities, such as but not limit to, monitoring warehouse traffic, communicating production (such as pick & pack) status and requirement, inventory management, etc.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

As used herein in reference to an element and a standard, the term “compatible” means that the element communicates with other elements in a manner wholly or partially specified by the standard, and would be recognized by other elements as sufficiently capable of communicating with the other elements in the manner specified by the standard. The compatible element does not need to operate internally in a manner specified by the standard.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. Signals and corresponding nodes or ports may be referred to by the same name and are interchangeable for purposes here.

It will be further understood that various changes in the details, materials, and arrangements of the parts which have been described and illustrated in order to explain the nature of this invention may be made by those skilled in the art without departing from the scope of the invention as expressed in the claims to be filed upon the USPTO utility and International filing.

### ABSTRACT OF THE DISCLOSURE

The described embodiments provide systems and methods for systems and platforms that receive data from and send data to wearable devices, receive data from environmental sensors, and visualization which collectively creates a kinematic chain allow for input from individuals, groups, third party information and interfaces with historical data as well as predictive modeling systems. Systems that include data from interoperable wearables in conjunction with one or more sensors and measuring devices may be used for the general public, corporations, employees, education, law enforcement, medical, sports, government, transportation, research and training, etc. Furthermore, the interoperability of one or more sensors and communication modules could be networked with one or more physical entities and objects creating an interactive communication network. Wearable platforms could include but are not limited to all wearables (inter- and intra- and proximity-wearables), smart devices, IT networks, virtual systems and secure information processes. In addition, the platforms and networks could also include real-time biological interoperability based on various synchronized and/or asynchronizated data inputs, information and media.

1. A system comprising at least one smart wearable which contains at least one sensor to record physical properties, include body movements, angle, speed, location and other physical and biodata, and at least one interface with a network capable of utilizing the information obtained from the at least one sensor.

2. The system of claim I wherein the network capable of utilizing the information obtained from the at least one sensor comprises one or more units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculation and algorithms, statistical, predictive, integrative, interpretative, exploratory, abnormality seeking, data producing, visualizing or presentation development platforms.

3. The system of claim I wherein the network comprises a system of tracking body movement or condition integrated with an analytical or predictive capacity to determine or estimate possible points or origin, routes of travel or proximity to specific event or locations.

4. The system of claim I where in the network comprises transmission through WiFi or other wireless modes.

5. The system of claim I, further comprising a two-way communication for communicating the in a form of an alerting to a remotely devices or other user.

6. The system of claim 1, wherein the identifying of the pattern in the at least one bioparameter or physical movements involves identifying said patterns in at least one accelerometer-measured bioparameter or physical movements and identifying patterns in at least one non-accelerometermeasured bioparameter or physical movements.

7. A method for obtaining sensor data from at least one wearable device, the method comprising the steps of implanting a smart wearable device capable of providing information relevant to the body movement or team coordination, to obtain the information relevant to the health of the animal or human, and transmitting at least some portion of the physical information to a network capable of utilizing the information obtained.

8. The method claim 7 wherein the physical property is transmitted securely to a plurality of remote devices monitoring the body movements and team coordination.

9. The method claim 7 wherein the physical property is transmitted securely to a plurality of remote devices monitoring a plurality of body movements.

10. The method claim 7 wherein the network capable of utilizing the information obtained from the at least one wearable device comprises one or more having the function of data storage, data retrieval, data synthesis, alert programs, data management, characterization, filtering, transformation, sorting, processing, modeling, mining, inspecting, investigation, retrieval, integrating, dissemination, qualitative, normalizing, clustering, correlations, computer derived values and ranges, simple or complex mathematical calculations, statistical, predictive, integrative, interpretative, exploratory, abnormality seeking, data producing, visualizing or presentation development platforms.

11. A team sport diagnostic system comprising a smart device which is attached to one or more team members, at least one wearable device attached to one or more team members, the device in turn being configured to obtain information from the at least one wearable device from one or more team members and to remotely transmit the information from the wearable sensor to an network for analysis.

12. The diagnostic system of claim 11 wherein the network interfaces with a mobile device worn or carried by a user of sensor information.

13. The diagnostic system of claim 11 wherein the device could be a wrist band, mouth guard, hat, pin, watch, glasses, cloth, shoes, socks, gloves, or any wearable device could be attached to the part of body.

14. The diagnostic system of claim 11 wherein the wearable measurements could be physical data or biological data include but not limit to oxygen level, heart rate, physical movement, angle of movement, etc.

15. The diagnostic system of claim 11 wherein the network is configured to analyze fatigue of an individual team member.

16. The diagnostic system of claim 11 wherein the network is configured to analyze fatigue of a composite of a plurality of team members.

17. The diagnostic system of claim 11 wherein the system is configured to give user of mobile device information feedback regarding a physiological characteristic of a current activity.

18. The diagnostic system of claim 11 wherein the current activity is selected from the group consisting of running, jogging, walking, sleeping, and a physical characteristic of playing a sport.

19. A system for the integration of wearables, comprising: a) individual wearable devices, or a network of wearable devices, individually or as a team; (b) wearables could be implantable or attached to any part of body; (c) can measure bioparameters or exactly physical movements of the person or part of the body: (d) wearable output data from the one or more wearables concerning the measured bioparameters or physical activity and; (e) local processors configured to collect and communicate specific data.
